(19) United States
(12) Patent Application Publication
(10) Pub. No.: US 2015/0083590 A1
(43) Pub. Date: Mar. 26, 2015

(54) PORTABLE DEVICE ASSEMBLY FOR MEASURING BLOOD SAMPLES AND A DEVICE COVER

(71) Applicants: Carina Rajala, Helsinki (FI); Salla Koski, Helsinki (FI); Mikko Tasanen, Helsinki (FI)

(72) Inventors: Carina Rajala, Helsinki (FI); Salla Koski, Helsinki (FI); Mikko Tasanen, Helsinki (FI)

(73) Assignee: Modz Oy, Helsinki (FI)

(21) Appl. No.: 14/387,041

(22) PCT Filed: Mar. 23, 2012
(86) PCT No.: PCT/FI2012/050295

§ 371 Date: Sep. 22, 2014

(51) Int. Cl. G01N 27/28; G01N 33/487; G01N 33/66

(52) U.S. Cl. CPC G01N 27/28; G01N 33/66; G01N 33/48785
USPC 204/403.02

(57) ABSTRACT

A device assembly for measuring and monitoring glucose content of blood, and a cover of such an assembly. The assembly is especially applicable for users with diabetes disease. The assembly includes a resilient cover on a measurement device. The cover has a slit at the input for a blood sample strip. The slit is closed at its rest position, but a test strip can be entered through the slit based on the resilience of the cover material. The resilient cover absorbs possible mechanical impacts and prevents impurities from reaching the sensor electronics.

### FIELD OF INVENTION

The invention relates to a portable device assembly for measuring blood samples, such as glucose content of blood samples, and a device cover of the assembly. The invention is especially applicable for users who have diabetes disease.

### BACKGROUND TECHNOLOGY

A person with a diabetes disease must take especially good care for his/her diet, general condition, regular rest and medication. The care of the diabetes disease also demands regular concentration measurement of blood sugar i.e. blood glucose. The measurement must be performed even several times a day. The measurement commonly takes place by taking a small sample of blood from a finger tip, which sample is let to absorb into a sample test dot of a sample test strip. The glucose concentration is measured from the strip with a portable measurement device, for example. Such a device according to prior art is described e.g. in publication WO2008/62099A1. A measurement device is used by placing a sample test strip to the measurement device and setting a blood sample on the test strip, after which the measurement device performs blood glucose measurement from the blood sample, and shows the measurement result on the display of the measurement device.

There are some problems related to such prior art devices. The blood sample is measured by detecting an electric charge, which is created by a chemical reaction. The measurement unit the reads the electric current profile created by the charge. It is therefore important that the electrical contacts of the measurement unit remain clean from impurities. The measurement unit may also read optically calibration data from the sample strip, whereby the optics must also remain clean. Further, the sample strips are sensitive to moisture. Therefore, a measurement device must normally be kept in a specific bag or box. This means that it is often difficult carry the measurement device along. As a consequence, it is possible that the user does not always have the device when it is needed. On the other hand, if the device is kept in a pocket, for example, impurities cumulated in the measurement unit may cause the device to give inaccurate readings.

Since diabetes is a common disease, insufficient care is a significant problem for the person himself/herself but also for the society. Therefore, the improvement of care and solving the above disadvantages of the prior art devices is important.

### SUMMARY OF THE INVENTION

The object of the invention is to provide a device assembly for measuring blood samples and a device cover of the assembly, by which solution the above problems can be decreased or avoided.

The object of the invention can be achieved with a user portable measurement device assembly, which comprises a measurement unit for measuring a blood sample for e.g. blood glucose concentration, wherein the measurement unit has an input for providing the blood sample strip in the measurement unit, and the device has a user interface for presenting to a user information which is based on a measurement result, and which assembly is characterised in that the assembly comprises a resilient cover for the measurement device which cover has a slit at the input for the blood sample strip, and the slit is at least as wide as the width of the blood sample strip, whereby the slit is arranged to be substantially closed in its rest position, and allows a blood sample strip to be entered through the slit based on the resilience of the cover material.

The object of the invention can also be achieved with a cover for a user portable measurement device for measuring a blood sample for e.g. blood glucose concentration, wherein the measurement device has an input for providing the blood sample strip in the measurement device, and the device has a user interface for presenting to a user information which is based on a measurement result, and which is characterised in that the cover is made of resilient material, the cover has a slit at the input for the blood sample strip, and the slit is at least as wide as the width of the blood sample strip, whereby the slit is arranged to be substantially closed in its rest position, and allows a blood sample strip to be entered through the slit based on the resilience of the cover material.

Some preferable embodiments of the invention are described in the dependent claims.

According to one embodiment of the invention the resilient cover is made of silicone. The resilient cover may, however, be made of some other alternative material such as soft plastic of fabric.

According to a further embodiment the resilient cover is translucent. The measurement device may further have means for lighting the sample input, whereby the resilient cover is also illuminated at the area of the sample input. The input light may be switched ON when the device gives a reminder for measurement, for example.

According to one embodiment of the invention the resilient cover has a hook for a carrying strap/neckband.

The resilient cover according to the invention is preferably detachable from the device by stretching the resilient cover.

According to a further embodiment of the invention the measurement device has an electrical connector for e.g. charging or data transfer, which connector is covered by the resilient cover.

The present invention and its embodiments offer substantial advantages over prior art solutions. A cover according to the invention prevents impurities from reaching the measurement sensor and related electronics and optics. However, it is still easy to make a measurement of a blood sample as there is no need to open any bags boxes or covers. The device can be held in a pocket or it may be worn in a neckband. Therefore, it easy to always carry the device along. This makes it possible for the user to perform required measurements and possible other advised care whenever needed. Optimal care is thus achieved. A resilient cover provides a good shield against impacts, for example, if the device is accidentally dropped. A resilient cover is also pleasant and ergonomical for a user.

In this patent application “measurement device” means a device for measuring blood samples, such as glucose concentration and/or other quantities. A measurement device may also provide monitoring functions, reminder functions, data processing functions, data transfer functions, database functions, game functions etc.

“A sample strip” means in this patent application e.g. a strip, which has blood sample test dots for absorption and measurement of a blood sample.

“A user of a measurement device” means in this patent application primarily a person, whose blood samples are measured with the measurement device.

### BRIEF DESCRIPTION OF DRAWINGS

In the following the invention is described with reference to the enclosed drawings, in which:

FIG. 1 illustrates a back view of the internal layout of an exemplary measurement device;

FIG. 2a illustrates a perspective view of an exemplary measurement device;

FIG. 2b illustrates a bottom view of an exemplary measurement device;

FIG. 3a illustrates a perspective view of an exemplary measurement device assembly according to the invention; and

FIG. 3b illustrates a perspective view of an exemplary measurement device assembly according to the invention; and

FIG. 3c illustrates a perspective view of an exemplary measurement device assembly according to the invention; and

FIG. 3d illustrates a perspective view of an exemplary measurement device assembly according to the invention; and

### DETAILED DESCRIPTION OF PREFERRED EMBODIMENTS

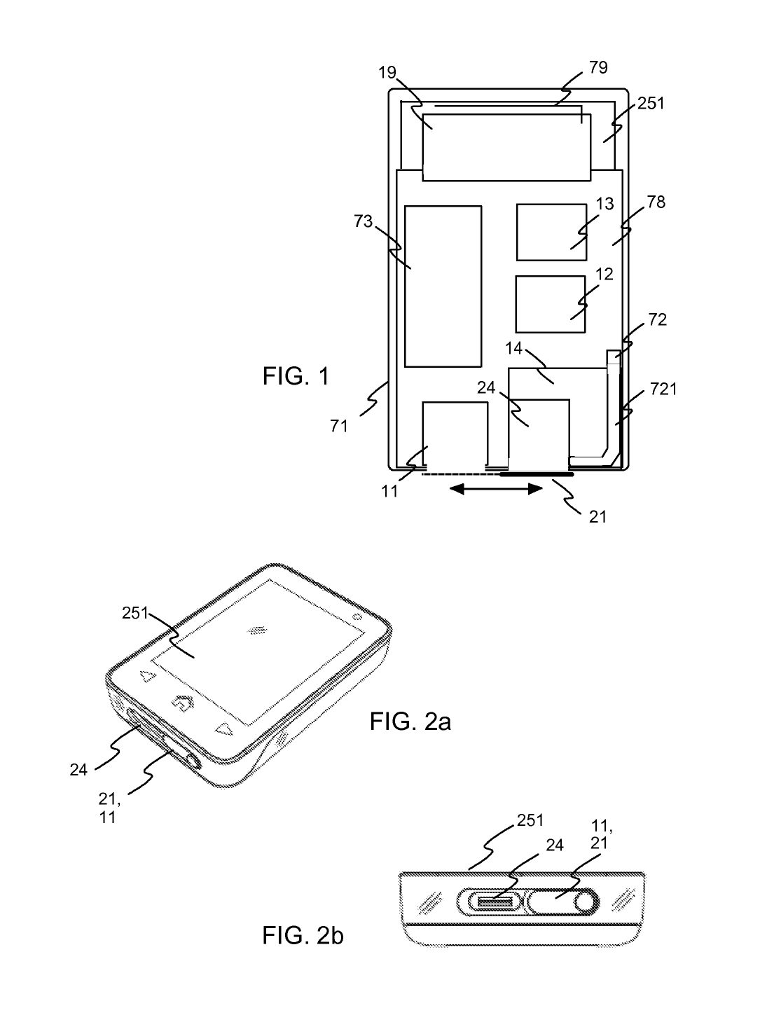

FIG. 1 illustrates a back view of the internal layout of an exemplary measurement device, which may be used in an assembly according to the invention. The measurement device has an electronics module 78, which has the touch screen 251 at its front side, and other units/components at the back side. The measurement unit 14 is located at the bottom of the device. The measurement unit has an input aperture 24 at the bottom side of the device for placing a test strip, on which a blood sample is then absorbed. The measurement unit is preferably based on electrical charge measurement in a manner, which is prior known as such. The measurement unit preferably includes an amplifier, an analogue-digital converter and other required electronics so that the signal received from the sensor can be fed to the input of a main processor 12 of the device. The processor preferably saves the measurement results into a memory 13 for later use. Also programs 16 controlling the processor have been stored into the memory 13 of the measurement device. A wireless communication unit 19 and its antenna 79 are located at the top area of the device.

An electrical USB connector 11 is located at the bottom side of the device, besides the sample strip input. The USB connector can be used for charging a rechargeable battery 73 of the device and for transferring data, for example. The device has a sliding lid 21, which can be moved to cover either the electrical connector or the sample input.

The device has also a light emitter, such as an LED 72, which is preferably controlled by the processor 12. The light from the LED is conveyed to the sample input aperture with a light guide 721. The light guide makes it possible to provide an optimal light distribution at the aperture 24. FIG. 1 also illustrates the processor 12, memory 13 and a battery 73.

FIGS. 2a and 2b illustrate a perspective view and a bottom view of an exemplary measurement device, which can be a part of a measurement device assembly according to the invention, and which can thus be used with a resilient cover according to the invention. The sample input 24 and the USB connector 11 are located at the bottom side of the device. There may be a sliding lid 21, which can be moved to cover either the sample input 24 or the electrical connection 11. The purpose the sliding lid is to prevent simultaneous use of the electrical connection and sample measurement.

The measurement device of FIGS. 1, 2a and 2b also has a touch screen 251 as a user interface. The measurement device may present e.g. measurement results, instructions and reminders at the touch screen, and the reminders can be acknowledged by touching the touch screen. It is also possible to use tones or recorded/synthesized speech or vibration for reminding and instructing the user.

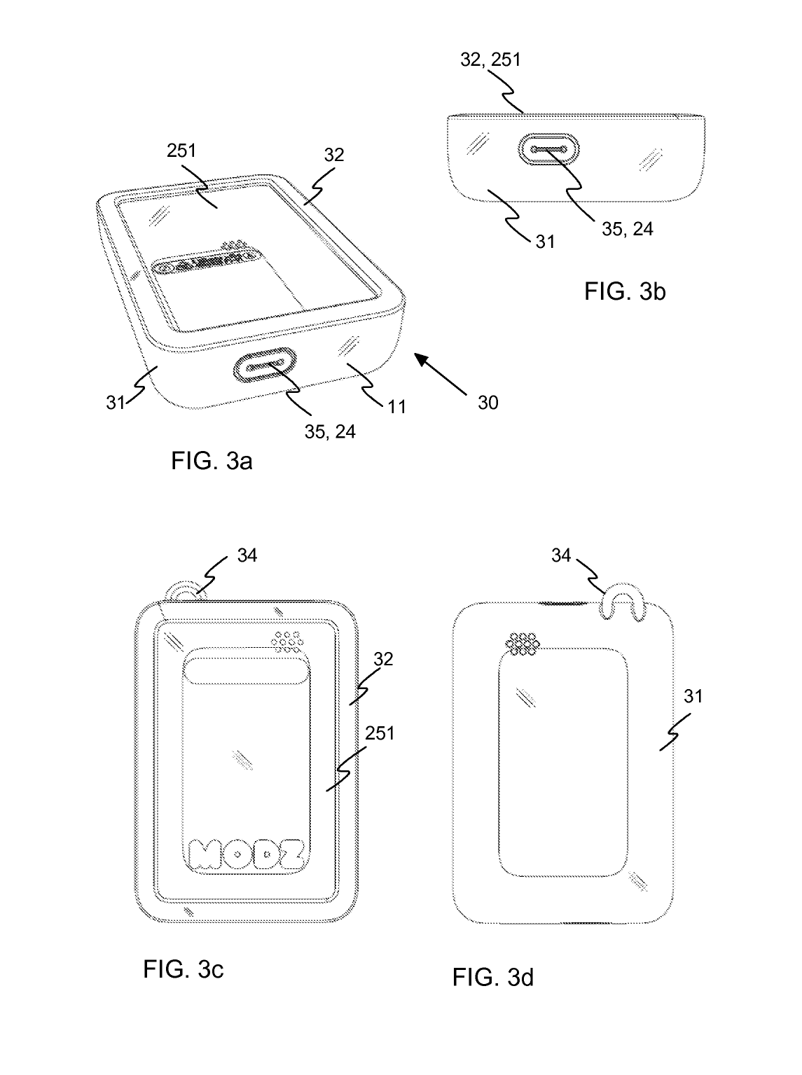

FIGS. 3a-3d illustrate an exemplary measurement device assembly 30 according to the invention. FIG. 3a shows a perspective view, FIG. 3b shows a bottom view, FIG. 3c shows a front view, and FIG. 3d shows a back view of the assembly.

The assembly 30 has a resilient cover 31 on a measurement device. The measurement device may be a device according to FIGS. 1, 2a and 2b, for example. The resilient cover is preferably made of silicone, but it may also be made of some other material with corresponding properties, such as soft plastic or fabric. The cover preferably has a tight fitting on the device, and the cover may be detached by stretching the cover. The cover may also be made of materials with various colours. The cover or part of it may also be made of translucent material whereby the area of the sample strip input can be illuminated with a light.

The cover has edges 32 at the front of the device, remaining the touch screen 251 or other kind of user interface open and available for use. The cover may also have a hook 34 for attaching a carrying strap/neckband. The cover may be closed at the back side of the device or it may have an opening at the back.

In accordance with the invention the resilient cover has a slit 35 in front of the sample strip input 24 of the measurement device. The slit is substantially closed in its rest position in order to prevent impurities from reaching the measurement sensor, electronics and related optics. However, based on the resilience of the cover material it is possible to enter a sample strip through the slit into the sample strip input of the measurement unit of the measurement device.

It must be noted that above only some embodiments of the solution according to the invention have been described. The principle of the invention can naturally be modified within the scope of protection determined by the patent claims, e.g. in details of implementation and areas of use.

The present invention is also not restricted to care of diabetes and monitoring of blood glucose concentration, but the invention can also be used for monitoring other diseases or for monitoring health. As an example, monitoring blood haemoglobin of athletes or people suffering from anaemia can be mentioned.

The present invention is neither restricted to use of the materials, forms or functions which were described in the above embodiments, but a person skilled in the art can design several alternative implementations in the frame of the inventive features.

1-11. (canceled)

12. A user portable measurement device assembly (30), which comprises a measurement unit (14) for measuring a blood sample for e.g. blood glucose concentration, wherein the measurement unit has an input (24) for providing the blood sample strip in the measurement unit, and the device has a user interface (251) for presenting to a user information which is based on a measurement result, wherein the assembly comprises a resilient cover (31) for the measurement device which cover has a slit (35) at the input (24) for the blood sample strip, and the slit is at least as wide as the width of the blood sample strip, whereby the slit is arranged to be substantially closed in its rest position, and allows a blood sample strip to be entered through the slit based on the resilience of the cover material.

13. A measurement device assembly according to claim 12, wherein the resilient cover (31) is made of silicone, plastic or fabric.

14. A measurement device assembly according to claim 12, wherein at least part of the resilient cover (31) is made of translucent material.

15. A measurement device assembly according to claim 12, wherein the measurement device has means (72, 721) for lighting the sample input, whereby the resilient cover (31) is illuminated at the area of the sample strip input (24, 35).

16. A measurement device assembly according to claim 12, wherein the resilient cover has a hook (34) for a carrying strap/neckband.

17. A measurement device assembly according to claim 12, wherein the resilient cover (31) is detachable from the device by stretching the resilient cover.

18. A measurement device assembly according to claim 12, wherein the measurement device has an electrical connector (11), which is covered by the resilient cover (31).

19. A cover (31) for a user portable measurement device for measuring a blood sample for e.g. blood glucose concentration, wherein the measurement device has an input (24) for providing the blood sample strip in the measurement device, and the device has a user interface (251) for presenting to a user information which is based on a measurement result, wherein the cover (31) is made of resilient material, the cover has a slit (35) at the input for the blood sample strip, and the slit is at least as wide as the width of the blood sample strip, whereby the slit is arranged to be substantially closed in its rest position, and allows a blood sample strip to be entered through the slit based on the resilience of the cover material.

20. A cover according to claim 19, wherein the resilient cover (31) is made of silicone, plastic or fabric.

21. A cover according to claim 19, wherein at least part of the resilient cover (31) is made of translucent material.

22. A cover according to claim 19, wherein the resilient cover has a hook (34) for a carrying strap/neckband.
